(19) United States
(12) Patent Application Publication
(10) Pub. No.: US 2024/0374197 A1
(43) Pub. Date: Nov. 14, 2024

(54) CATEGORIZATION AND ANALYSIS OF LOW SIGNAL QUALITY IN ECG DATA

(71) Applicant: Boston Scientific Cardiac Diagnostics, Inc., Rochester, MN (US)

(72) Inventors: Joseph F. Lambrecht, Byron, MN (US); David R. Engebretsen, Cannon Falls, MN (US); Timothy P. McClanahan, Rochester, MN (US); Michael Thomas Edward McRoberts, Mazeppa, MN (US); Riley Ellis, Rochester, MN (US)

(21) Appl. No.: 18/656,973

(22) Filed: May 7, 2024

Related U.S. Application Data
(60) Provisional application No. 63/464,808, filed on May 8, 2023.

(51) Int. Cl. A61B 5/00; G16H 40/60

(52) U.S. Cl. CPC A61B 5/352; A61B 5/7221; G16H 40/60; A61B 5/0006

(57) ABSTRACT

Systems of methods involve receiving a package of data comprising strips of electrocardiogram (ECG) data and metadata; displaying, on a user interface, at least some of the strips of ECG data, including a strip of ECG data associated with an initial maximum heart rate; and overriding the initial maximum heart rate in response to selecting a strip of ECG data associated a lower maximum heart rate.

### CROSS REFERENCE TO RELATED APPLICATION

This application claims priority to Provisional Application No. 63/464,808, filed May 8, 2023, which is herein incorporated by reference in its entirety.

### TECHNICAL FIELD

The present disclosure relates to devices, methods, and systems for analyzing cardiac activity and cardiac events.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes: receiving a package of data relating to a patient study. The package of data includes strips of electrocardiogram (ECG) data and metadata, which includes an initial maximum heart rate of the patient study. The method further includes displaying, on a user interface, at least some of the strips of ECG data, including a strip of ECG data associated with the initial maximum heart rate. The method further includes overriding the initial maximum heart rate in response to selecting a strip of ECG data comprising a lower maximum heart rate.

In Example 2, the method of Example 1, wherein the overriding the initial maximum heart rate is performed in response to: receiving, via the user interface, a user selection of the strip of ECG data comprising the lower maximum heart rate.

In Example 3, the method of any of the preceding Examples, wherein the strip of the ECG data with the initial maximum heart rate is associated with a noisier signal than the strip of ECG data comprising the lower maximum heart rate.

In Example 4, the method of any of the preceding Examples, wherein the overriding includes inserting a noisy signal flag into the strip of ECG data associated with the initial maximum heart rate.

In Example 5, the method of Example 4, wherein the noisy signal flag causes the strip of ECG data associated with the initial maximum heart rate to be removed as a candidate for containing a maximum heart rate.

In Example 6, the method of any of the preceding Examples, wherein the overriding the initial maximum heart rate comprises overriding all calculated maximum heart rates that are greater than the lower maximum heart rate.

In Example 7, the method of any of the preceding Examples, wherein the metadata includes an initial minimum heart rate of the patient study. The method further includes overriding the initial minimum heart rate in response to selecting a strip of ECG data comprising a higher minimum heart rate.

In Example 8, the method of any of the preceding claims, wherein the initial maximum heart rate and the lower maximum heart rate are heart rates calculated during respective most acute sections of the ECG data.

In Example 9, the method of Example 8, wherein the respective most acute sections comprise no more than 10 seconds of the ECG data.

In Example 10, the method of any of Examples 8 or 9, wherein the respective most acute sections comprise no more than 6 beats within the ECG data.

In Example 11, the method of any of Examples 8-10, wherein the heart rates are average heart rates of the most acute sections.

In Example 12, the method of any of the preceding Examples, wherein the package of data includes strips of ECG data associated with a noisy signal. The method further includes excluding the strips of ECG data associated with a noisy signal as a candidate for containing a maximum heart rate.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a system includes a remote computing system with: a user interface, one or more processors, and computer-readable media having computer-executable instructions embodied thereon. The instructions are configured to be executed by the one or more processors to cause the remote computing system to: after receiving a package of data comprising strips of ECG data and metadata, which includes an initial maximum heart rate of the patient study: display on the user interface at least some of the strips of ECG data, including a first strip of ECG data associated with the initial maximum heart rate and a second strip of ECG data. The instructions are further configured to be executed by the one or more processors to cause the remote computing system to: receive a user selection of the second strip, the second strip of ECG data associated with a lower maximum heart rate; and override the initial maximum heart rate in response to the user selection.

In Example 17, the system of Example 16, wherein the strip of the ECG data associated with the initial maximum heart rate is associated with a noisier signal than the strip of ECG data comprising the lower maximum heart rate.

In Example 18, the system of Example 16, wherein the overriding includes inserting a noisy signal flag into the strip of ECG data associated with the initial maximum heart rate.

In Example 19, the system of Example 18, wherein the noisy signal flag causes the strip of ECG data associated with the initial maximum heart rate to be removed as a candidate for containing a maximum heart rate.

In Example 20, the system of Example 16, wherein the override the initial maximum heart rate comprises overriding all calculated maximum heart rates that are greater than the lower maximum heart rate.

In Example 21, the system of Example 16, wherein the metadata includes an initial minimum heart rate, wherein the instructions are configured to be executed by the one or more processors to cause the remote computing system to: override the initial minimum heart rate in response to selecting a strip of ECG data comprising a higher minimum heart rate.

In Example 22, the system of Example 16, wherein the initial maximum heart rate and the lower maximum heart rate are heart rates calculated during respective most acute sections of the ECG data.

In Example 23, the system of Example 22, wherein the heart rates are average heart rates of the most acute sections.

In Example 24, the system of Example 22, wherein the respective most acute sections comprise no more than 6 beats within the ECG data or no more than 10 seconds of ECG data.

In Example 25, the system of Example 16, wherein the first set of instructions are part of the package of data received from a server.

In Example 26, the system of Example 25, wherein the first set of instructions are executed by a web browser at the remote computing system.

In Example 27, the system of Example 16, wherein the package of data includes strips of ECG data associated with a noisy signal, wherein the instructions are configured to be executed by the one or more processors to cause the remote computing system to: exclude the strips of ECG data associated with a noisy signal as a candidate for containing a maximum heart rate.

In Example 28, a method includes: receiving a package of data comprising strips of ECG data and metadata, the metadata including an initial maximum heart rate of the patient study; displaying, on a user interface, at least some of the strips of ECG data, including a strip of ECG data associated with the initial maximum heart rate; and overriding the initial maximum heart rate in response to selecting a strip of ECG data associated a lower maximum heart rate.

In Example 29, the method of Example 28, wherein the overriding the initial maximum heart rate is performed in response to: receiving, via the user interface, a user selection of the strip of ECG data comprising the lower maximum heart rate.

In Example 30, the method of Example 28, wherein the overriding the initial maximum heart rate comprises overriding all calculated maximum heart rates that are greater than the lower maximum heart rate.

In Example 31, the method of Example 28, wherein the metadata includes an initial minimum heart rate, the method further includes: overriding the initial minimum heart rate in response to selecting a strip of ECG data comprising a higher minimum heart rate.

In Example 32, the method of Example 28, wherein the initial maximum heart rate and the lower maximum heart rate are heart rates calculated during respective most acute sections of the ECG data.

In Example 33, the method of Example 32, wherein the respective most acute sections comprise no more than 10 seconds of the ECG data.

In Example 34, the method of Example 32, wherein the respective most acute sections comprise no more than 6 beats within the ECG data.

In Example 35, the method of Example 32, wherein the heart rates are average heart rates of the most acute sections.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, remote computer, and user interface, in accordance with certain instances of the present disclosure.

FIGS. 3-5 show various screenshots of a user interface, in accordance with certain instances of the present disclosure.

FIGS. 6 and 7 show block diagrams depicting illustrative methods, in accordance with instances of the disclosure.

FIG. 8 shows a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

The present disclosure relates to devices, methods, and systems for facilitating analysis of cardiac activity and cardiac events such as abnormal cardiac rhythms or other heart-related issues.

Electrocardiogram (ECG) data of a patient can be used to monitor cardiac activity and/or identify whether the patient has experienced a cardiac event. For example, a monitoring device attached to a patient can be used to produce signals that represent the patient's cardiac activity in the form of ECG data. The patient's ECG data can be collected over one or more days as part of a physician-directed study of the patient's cardiac activity. The ECG data from the study can be used to detect abnormal cardiac rhythms as well as calculate key metrics of the patient's cardiac activity such as heart rates (e.g., maximum and minimum heart rates) and RR intervals (e.g., longest RR interval), and the like.

However, over the course of the study, there may be time periods where the signals produced by the monitoring device contain noise, which results in low signal quality. For example, there may be moments where the monitoring device experiences interference or has less than ideal coupling with the patient which introduces noise into the sensing signals. Noisy signals can cause various cardiac-related calculations to be inaccurate which therefore should be ignored. As examples, a calculated maximum heart rate and/or minimum heart rate may be inaccurate during such time periods with noisy signals. Certain instances of the present disclosure are accordingly directed to systems, methods, and devices for addressing low signal quality.

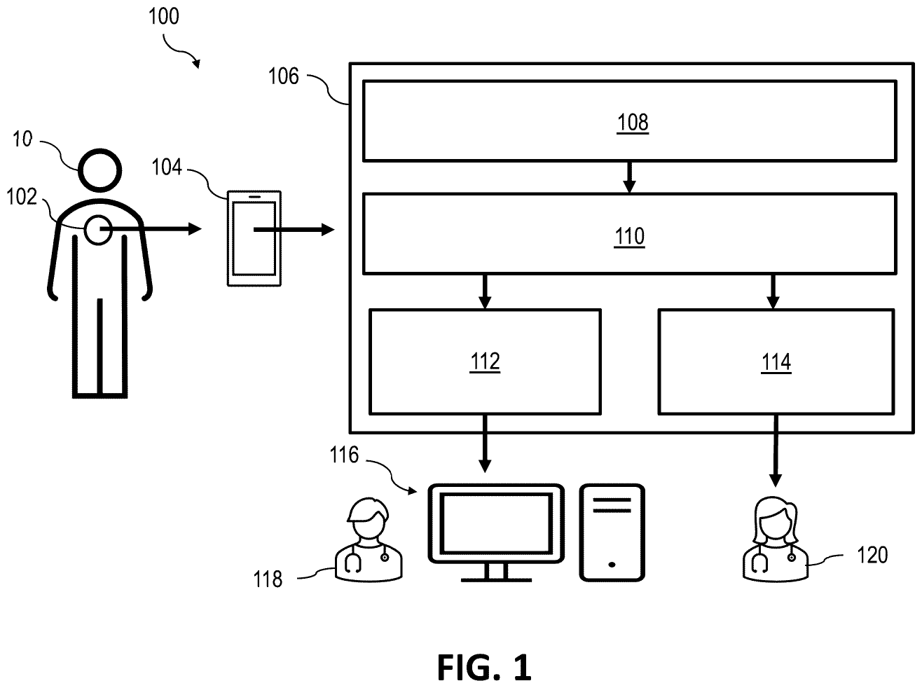

FIG. 1 illustrates a patient 10 and an example system 100. The system 100 includes a monitor 102 attached to the patient 10 to detect cardiac activity of the patient 10. The monitor 102 may produce electric signals that represent the cardiac activity in the patient 10. For example, the monitor 102 may detect the patient's heart beating (e.g., using infrared sensors, electrodes) and convert the detected heartbeat into electric signals representing ECG data. The monitor 102 communicates the ECG data to a mobile device 104 (e.g., a mobile phone).

The mobile device 104 may include a program (e.g., mobile phone application) that receives, processes, and analyzes the ECG data. For example, the program may analyze the ECG data and detect or flag cardiac events (e.g., periods of irregular cardiac activity) contained within the ECG data. Because ECG data may be getting continuously generated, the amount of ECG data can be overwhelming to store and process locally on the mobile device 104. As such, the mobile device 104 can periodically transmit chunks of the ECG data to another device or system, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104. Also, in certain instances, the monitor 102 and/or the mobile device 104 includes a button or touch-screen icon that allows the patient 10 to initiate an event. Such an indication can be recorded and communicated to the other device or system.

### Cardiac Event Server

In the example shown in FIG. 1, the mobile device 104 transmits the ECG data (and associated metadata, if any) to a cardiac event server 106 (hereinafter “the server 106” for brevity). The server 106 includes multiple platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108 (e.g., deep neural networks), a cardiac event router 110, a report platform 112, and a notification platform 114. Although only one server 106 is shown in FIG. 1, the server 106 can include multiple separate physical servers, and the various platforms/modules/layers can be distributed among the multiple servers. Each of the platforms/modules/layers can represent separate programs, applications, and/or blocks of code where the output of one of the platforms/modules/layers is an input to another of the platforms/modules/layers. Each platform/module/layer can use application programming interfaces (APIs) to communicate between or among the other platforms/modules/layers as well as systems and devices external to the server 106.

The server 106 applies the machine learning model 108 to the ECG data to determine various cardiac data and events. As examples, the machine learning model 108 may determine the patient's heart rate (including a maximum heart rate) over the course of the study and individual heart rates (including individual maximum heart rates) over the course of different, shorter time intervals. As other examples, the machine learning model 108 may determine the patient's heart rate (including a minimum heart rate) over the course of the study and individual heart rates (including individual minimum heart rates) over the course of different, shorter time intervals. However, as noted above, because of noise in the ECG data at various time intervals, the machine learning model 108 may calculate heart rates that are not accurate.

In certain embodiments, the machine learning model 108 includes two paths, where the first path is a deep convolutional neural network and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The deep fully connected neural network receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model 108 determines that the ECG data most closely resembles a labeled ECG data associated with a cardiac event, then the machine learning model 108 may determine that the patient 10 has experienced that cardiac event. Additionally, the machine learning model 108 may measure or determine certain characteristics of the cardiac activity of the patient 10 based on the ECG data. For example, the machine learning model 108 may determine a heart rate, a duration, or a beat count of the patient 10 during the cardiac event based on the ECG data.

The machine learning model 108 may also determine a confidence level for each classification or identification of a cardiac event. The confidence level is an indication of certainty or uncertainty in the accuracy of the machine learning model's classification or identification. In certain instances, machine learning model 108 may identify the existence of beats but may not be able to classify the beats and/or classify a rhythm in which the beats are located. For example, the machine learning model 108 may determine that the calculated confidence level for such beats/rhythm is not high enough to provide classifications. For these situations, the time intervals that contain such unclassified beats/rhythms may be flagged as a low signal quality time interval. In other examples, the machine learning model 108 may analyze a signal-to-noise ratio (SNR) of the ECG data. Once “noisy” time intervals have been identified, the machine learning model 108 can generate metadata that indicates the start time and end time of such time intervals and an indicator or flag that indicates that the respective time intervals are low signal quality time intervals (or a noisy signal time intervals).

The server 106 stores the cardiac event (and associated metadata such as information like heart rate, duration, beat count, low signal quality, etc.) in a database for storage. Subsequently, the server 106 may retrieve the cardiac event and associated information from the database.

In certain instances, once the ECG data is processed by the machine learning model 108, the ECG data is made available for the report platform 112. As will be described in more detail below, the report platform 112 can be accessed by a remote computer 116 (e.g., client device such as a laptop, mobile phone, desktop computer, and the like) by a user at a clinic or lab 118.

In other instances, the cardiac event router 110 is used to determine what platform further processes the ECG data based on the classification associated with the cardiac event. For example, if the identified cardiac event is severe, the cardiac event router 110 can flag or send the ECG data, etc., to the notification platform 114. The notification platform 114 can be programmed to send notifications (along with relevant ECG data and associated metadata) immediately to the patient's physician/care group remote computer 116 and/or to the patient 10 (e.g., to their computer system, e-mail, mobile phone application).

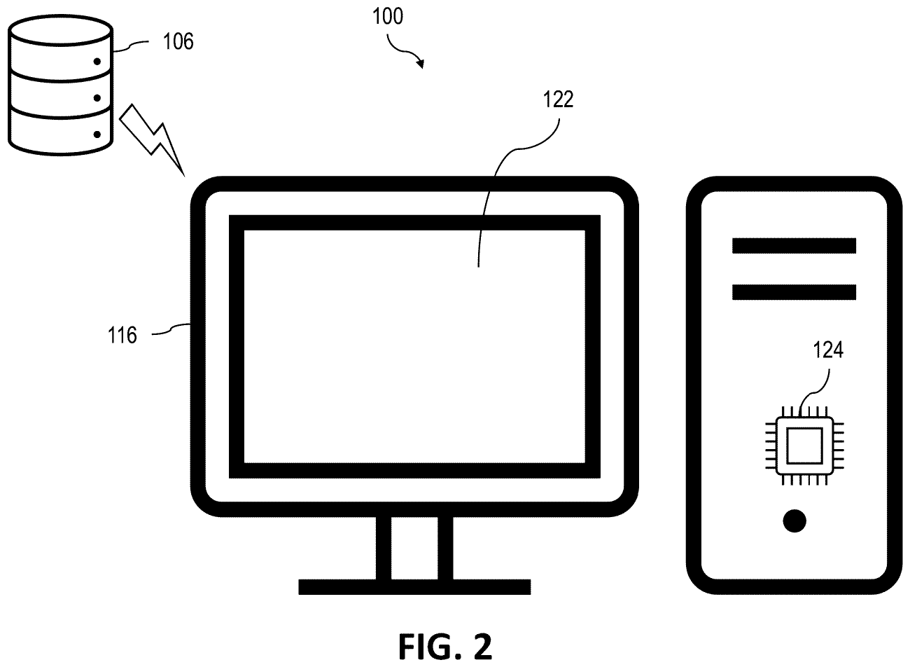

FIG. 2 shows the server 106 communicatively coupled (e.g., via a network) to the remote computer 116. In the example of FIG. 2, the remote computer 116 includes a monitor showing a user interface 122 that displays features of the report platform 112 hosted by the server 106. The Ul 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the user interface 122 to log into the report platform 112 via a web browser such that the user can use and interact with the report platform 112. When the user at the clinic or lab 118 is ready to analyze ECG data of a patient, the user can select a patient's profile through the user interface 122.

The server 106 (e.g., via programming associated with the report platform 112) can start a process for sending data to the remote computer 116. This data includes the ECG data and metadata associated with the ECG data. As noted above, once the ECG data from the monitored patients has been collected, the machine learning model 108 may determine certain characteristics of the cardiac activity of the patient 10 based on the ECG data, including estimating that a cardiac event has occurred and associating or generating metadata for the determined events. The metadata can include information about the patient 10, a heart rate of the patient 10 during the cardiac event, a duration of the cardiac event, a beat count of the cardiac event, a confidence level of the machine learning model's identification of the cardiac event, and/or a beat classification (e.g., normal, ventricular, supraventricular, unclassified). In certain instances, the machine learning model 108 attempts to assign each beat with a beat classification and, for certain groups and patterns of beats, a cardiac event type (e.g., atrial fibrillation, ventricular tachycardia, flutter). To distinguish among the beats, each individual beat can therefore be assigned a unique identifier (e.g., a unique number).

Accessing, processing, and displaying one or more days' worth of ECG data and metadata can consume a large amount of computing resources, network bandwidth resources, and human resources. To help alleviate burdens on these resources, the server 106 (e.g., via the report platform 112) can selectively transmit packages of ECG data and metadata to the remote computer 116.

The initial packages of data can include: (1) certain strips of ECG data including and/or surrounding detected cardiac events (e.g., 5-10 minutes strips of raw ECG data), (2) metadata associated with the strips, and (3) executable code (e.g., JavaScript code).

### Cardiac Activity Analysis

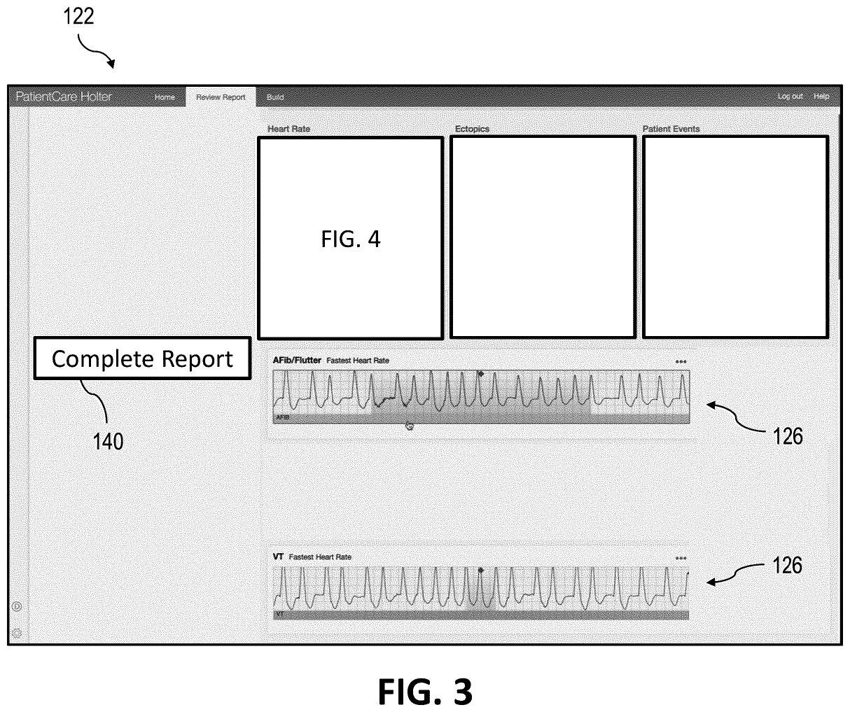

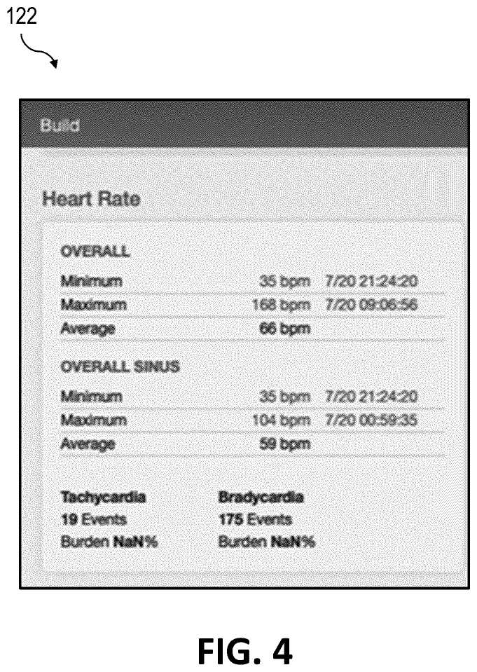

FIGS. 3 and 4 show different screenshots of the user interface 122, which can be used to generate a report that summarizes a patient's cardiac activity during a study. Examples of finalized reports include reports that are sometimes referred to as Holter reports (e.g., short-term Holter reports and long-term Holter reports) or cardiac event reports (e.g., MCT reports), among others.

In the view shown in FIG. 3, the user interface 122 displays a baseline summary of the patient's cardiac activity, which is based on ECG data collected during the patient study and processed at the server 106. For example, the user interface 122 can include a summary of heart rate information (shown in more detail in FIG. 4), ectopics, and events—each of which may have been initially been generated by the machine learning model 108. For a given type of event, the user interface 122 can display one or more example plots 126 of ECG data. In the example of FIG. 3, one of the plots 126 is the plot that contains the fastest heart rate during an atrial fibrillation event and another one of the plots 126 is the plot that contains the fastest heart rate during a ventricular tachycardia event.

FIG. 4 shows the user interface 122 displaying various types of heart rate information. The heart rate information can include, for example, the minimum overall heart rate of the study (and the start time of that heart rate in the study), the maximum overall heart rate of the study (and the start time of that heart rate in the study), the average overall heart rate of the study. Other examples include minimum, maximum, and average heart rates (and start times) for subsets or shorter time intervals of the overall study.

In certain instances, the initial values displayed for the heart rates mentioned above are the values initially calculated by the server 106 (e.g., by the machine learning model 108). However, as noted above, over the course of the study, there may be time periods where the signals produced by the monitoring device contain noise. Although the machine learning model 108 is programmed and trained to identify such time periods, there may be strips of ECG data that contain noise that were not initially classified as being noisy or with low signal quality. As such, various cardiac activity calculations such as heart rate calculations may be based on strips associated with some level of noise.

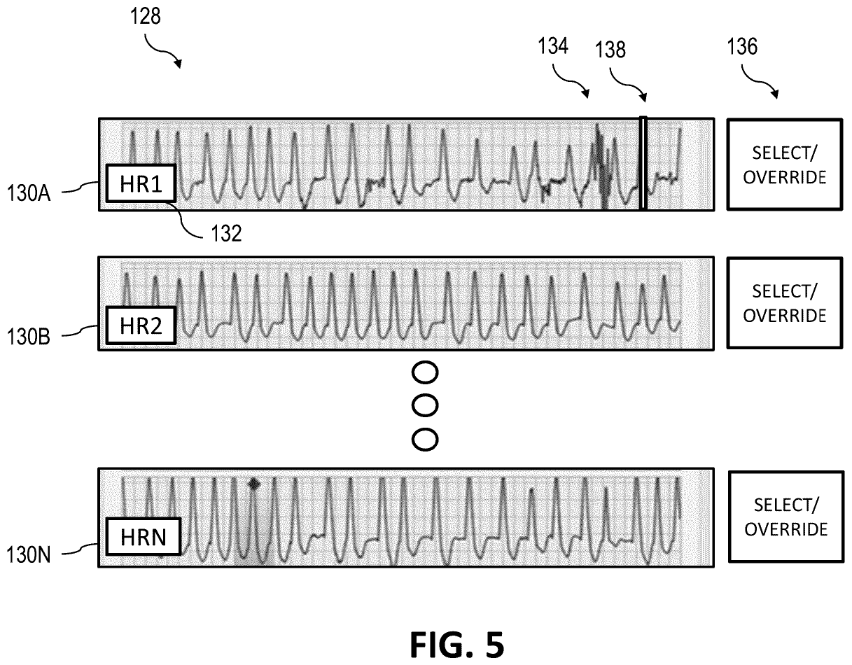

To help quickly identify and address such time intervals, the user interface 122 can display a heart rate summary page 128-a simplified example of which is shown in FIG. 5. In FIG. 5, the heart rate summary page 128 is shown as displaying multiple plots of strips of ECG data 130A, 130B, and 130N.

In certain instances, the strips of ECG data initially displayed on the heart rate summary page 128 are the strips that are associated with the fastest or slowest heart rates calculated (e.g., by the machine learning model 108) as part of the patient study. The heart rate summary page 128 can initially display strips of ECG data in descending order beginning with the fastest heart rate at the top (when analyzing the maximum heart rate) or beginning with the slowest heart rate at the top (when analyzing the minimum heart rate). Each strip of ECG data 130A to 130N displayed on the heart rate summary heart rate (or the minimum heart rate) for the respective strip of ECG data.

In certain instances, the maximum heart rate and the minimum heart rate are calculated based on identifying the most acute section within a portion of the ECG data (e.g., during a cardiac event detected by the one or more machine learning models 108). For example, during a detected cardiac event, the heart rates of the most acute sections can be calculated by identifying a subset of the cardiac event (e.g., an average over a multi-second section of ECG data such as 4-10 seconds or an average over a multi-beat section of ECG data such as a 3-6-beat section) with the fastest and slowest heart rate. In certain instances, the most acute sections can be sections with no more than 10 seconds of ECG data (or sections of no more than 6 beats) with the highest or lowest average heart rate. The heart rates of the most acute sections will be different than the overall average heart rate of a given cardiac event. For example, for a 1-minute cardiac event, the cardiac event might have an average over all of the beats of 80 beats per minute (BPM), but the most acute section (e.g., a 6-second section) may have an average of 100 BPM.

Using the heart rate summary page 128, a user can quickly scroll through and view the strips of ECG data with the fastest (or slowest) heart rates calculated by the machine learning model 108. This allows the user to view the underlying ECG data and determine whether the strips are associated with a noisy signal. For example, the strip of ECG data 130A at the top of FIG. 5 includes a segment 134 where there appears to be noise within the ECG data. Instead of having clear, distinct sections of a typical cardiac cycle in the plot of ECG data, the segment 134 includes what looks like many abrupt transitions between peaks and valleys in a short amount of time.

Once the user identifies which strip of ECG data should represent the maximum (or minimum) heart rate (e.g., heart rate during a most acute section of ECG data), the user can select that strip on the heart rate summary page 128. To select the strip of ECG data, the heart rate summary page 128 can include a selectable icon 136 (e.g., a button) next to each strip of ECG data. When the icon 136 associated with the designed strip of ECG data is selected, the heart rate associated with the selected strip can be designated as the maximum heart rate (or minimum heart rate) of the patient study. That maximum (or minimum) heart rate can then be used for generating a final report summary of the patient's cardiac activity.

In certain instances, in addition to selecting one of strips of ECG data for representing the maximum (or minimum) heart rate, the heart rate summary page 128 can be used to override heart rates that are greater (or less) than the selected heart rate. When the user selects one of the icons 136 from heart rate summary page 128, the strips of ECG data associated with faster heart rates (in the case of identifying a maximum heart rate) or slower heart rates (in the case of identifying a minimum heart rate) can be overridden. Put another way, strips with calculated heart rates (e.g., heart rate during the most acute sections) that are greater than the selected maximum heart rate or less than the selected minimum heart rate can be excluded as candidates for containing the maximum or minimum heart rate.

To exclude the strips from being candidates for containing the maximum or minimum heart rate, a flag or other feature can be inserted into (or otherwise associated with) the respective strips of ECG data. This flag can indicate (or otherwise label) the strip of ECG data as containing a noisy signal or low signal quality. For example, FIG. 5 shows the top strip of ECG data 130A as including a flag 138, which may indicate that the strip has a noisy signal or low signal quality. The flag can be the same type of flag used by the machine learning model 108 to indicate a noisy signal or low signal quality. As noted above, time intervals that contain unclassified beats/rhythms may be flagged by the machine learning model 108 as a low signal quality time interval. The same type and format of flag can be used for the strips of ECG data that have been overridden using the heart rate summary page 128. Because strips of ECG data that contain such a flag are excluded from maximum (or minimum) heart rate calculations, the overriding process described above can be used to quickly identify and flag which strips of ECG should be excluded from the calculations.

Using the above-described approach, strips of ECG data with the highest (or lowest) initial heart rate calculations can be efficiently analyzed and characterized and for purposes of determining which strips of ECG data should represent the maximum and minimum heart rates for a patient study. Instead of manually checking and correcting heart rates of each individual strip of ECG data, the approach described above can automate corrections, at least for calculated heart rates that are faster (or slower) than the maximum (and minimum) heart rates selected by the user. Once certain heart rates have been overridden, the system can update the summary data with the selected maximum and minimum heart rates.

To save processing and network resources and to allow these changes to occur in real-time, the calculations and changes to the maximum and minimum heart rates can be carried out locally on the remote computer 116—as opposed to sending large chunks of data back and forth between the server 106 and the remote computer 116. For example, the calculations can be carried out using cache memory 124 (shown in FIG. 1) and processing capabilities (e.g., one or more microprocessors) of the remote computer 116. To enable local processing and updating, the report platform 112 can send the remote computer 116 code to execute locally. This code uses (or operates on) the outputs of the machine learning model 108, which reduces the computational resources needed to process the changes made by user locally at the remote computer 116. In certain embodiments, this code is executed by an internet browser operating on the remote computer 116.

Once the user is satisfied with the simulated report, the user can select the “Complete Report” button 140 (shown in FIG. 3) in the user interface 122. This selection will start the process of generating a final report to be sent to the patient's physician.

In certain instances, once the report is built and complete, the remote computer 116 can send any changes to the metadata (e.g., the beat classifications and the rhythm classifications) to the server 106 and its database. The server 106 can then replace the metadata initially created by the machine learning model (and saved to the database) with the metadata generated by the remote computer while the user was reviewing and editing the metadata. As such, if the ECG and metadata need to be accessed again, the server's database has the most recent version of the metadata. Further, machine learning model may be further trained on the metadata generated by the user at the remote computer.

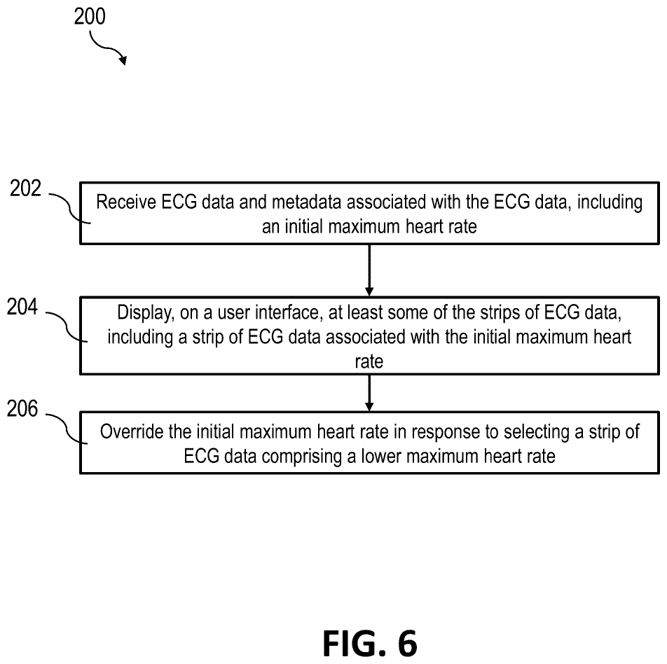

FIG. 6 shows a block diagram of a method 200 for overriding maximum heart rates initially calculated for a patient study. The method 200 includes receiving a package of data comprising ECG data and metadata (block 202 in FIG. 6). The metadata includes an initial maximum heart rate of the patient study. The method 200 further includes displaying, on a user interface, at least some of the strips of ECG data, including a strip of ECG data associated with the initial maximum heart rate (block 204 in FIG. 6). The method 200 further includes overriding the initial maximum heart rate in response to selecting a strip of ECG data comprising a lower maximum heart rate (block 206 in FIG. 6).

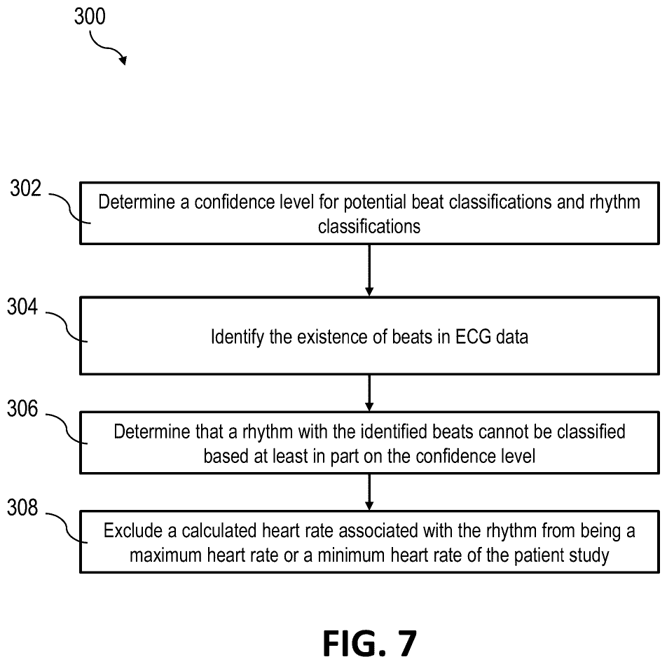

FIG. 7 shows a block diagram of a method 300 for excluding certain heart rate calculations as candidates for maximum or minimum heart rate determinations for a patient study. The method 300 includes determining, using a trained machine learning mode, a confidence level for potential beat classifications and rhythm classifications (block 302 in FIG. 7). The method 300 further includes identifying, by the trained machine learning model, the existence of beats within a strip of ECG data (block 304 in FIG. 7). The method 300 further includes determining, by the trained machine learning model, that a rhythm with the identified beats cannot be classified based at least in part on the confidence level (block 306 in FIG. 7). The method 300 further includes excluding a calculated heart rate associated with the rhythm from being a maximum heart rate or a minimum heart rate of the patient study (block 308 in FIG. 7).

### Computing Devices and Systems

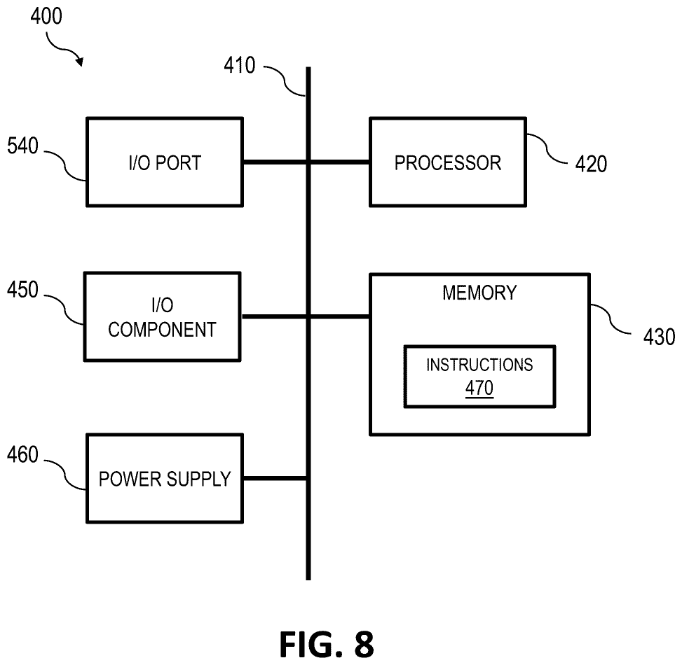

FIG. 8 is a block diagram depicting an illustrative computing device 400, in accordance with instances of the disclosure. The computing device 400 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 8 and described below. For example, the mobile device 104, the server 106, and the remote computer 116 can each include their own set of components shown in FIG. 8 and described below.

In instances, the computing device 400 includes a bus 410 that, directly and/or indirectly, couples one or more of the following devices: a processor 420, a memory 430, an input/output (I/O) port 440, an I/O component 450, and a power supply 460. Any number of additional components, different components, and/or combinations of components may also be included in the computing device 400.

The bus 410 represents what may be one or more busses (such as, for example, an address bus, data bus, or combination thereof). Similarly, in instances, the computing device 400 may include a number of processors 420, a number of memory components 430, a number of I/O ports 440, a number of I/O components 450, and/or a number of power supplies 460. Additionally, any number of these components, or combinations thereof, may be distributed and/or duplicated across a number of computing devices.

In instances, the memory 430 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 430 stores computer-executable instructions 470 for causing the processor 420 to implement aspects of instances of components discussed herein and/or to perform aspects of instances of methods and procedures discussed herein. The memory 430 can comprise a non-transitory computer readable medium storing the computer-executable instructions 470.

The computer-executable instructions 470 may include, for example, computer code, machine-useable instructions, and the like such as, for example, program components capable of being executed by one or more processors 420 (e.g., microprocessors) associated with the computing device 400. Program components may be programmed using any number of different programming environments, including various languages, development kits, and the like. Some or all the functionality contemplated may also, or alternatively, be implemented in hardware and/or firmware.

According to instances, for example, the instructions 470 may be configured to be executed by the processor 420 and, upon execution, to cause the processor 420 to perform certain processes. In certain instances, the processor 420, memory 430, and instructions 470 are part of a controller such as an application specific integrated circuit (ASIC), field-programmable gate array (FPGA), and/or the like. Such devices can be used to carry out the functions and steps described herein.

The I/O component 450 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. A system comprising:
a remote computing system comprising: a user interface, one or more processors, and computer-readable media having computer-executable instructions embodied thereon, the instructions configured to be executed to cause the remote computing system to:
after receiving a package of data comprising strips of electrocardiogram (ECG) data and metadata, which includes an initial maximum heart rate of the patient study: display on the user interface at least some of the strips of ECG data, including a first strip of ECG data associated with the initial maximum heart rate and a second strip of ECG data;
receive a user selection of the second strip, the second strip of ECG data associated with a lower maximum heart rate; and
override the initial maximum heart rate in response to the user selection.

2. The system of claim 1, wherein the strip of the ECG data associated with the initial maximum heart rate is associated with a noisier signal than the strip of ECG data comprising the lower maximum heart rate.

3. The system of claim 1, wherein the overriding includes inserting a noisy signal flag into the strip of ECG data associated with the initial maximum heart rate.

4. The system of claim 3, wherein the noisy signal flag causes the strip of ECG data associated with the initial maximum heart rate to be removed as a candidate for containing a maximum heart rate.

5. The system of claim 1, wherein the override the initial maximum heart rate comprises overriding all calculated maximum heart rates that are greater than the lower maximum heart rate.

6. The system of claim 1, wherein the metadata includes an initial minimum heart rate, wherein the instructions are configured to be executed by the one or more processors to cause the remote computing system to: override the initial minimum heart rate in response to selecting a strip of ECG data comprising a higher minimum heart rate.

7. The system of claim 1, wherein the initial maximum heart rate and the lower maximum heart rate are heart rates calculated during respective most acute sections of the ECG data.

8. The system of claim 7, wherein the heart rates are average heart rates of the most acute sections.

9. The system of claim 7, wherein the respective most acute sections comprise no more than 6 beats within the ECG data or no more than 10 seconds of ECG data.

10. The system of claim 1, wherein the first set of instructions are part of the package of data received from a server.

11. The system of claim 10, wherein the first set of instructions are executed by a web browser at the remote computing system.

12. The system of claim 1, wherein the package of data includes strips of ECG data associated with a noisy signal, wherein the instructions are configured to be executed to cause the remote computing system to: exclude the strips of ECG data associated with a noisy signal as a candidate for containing a maximum heart rate.

13. A method comprising:
receiving a package of data comprising strips of electrocardiogram (ECG) data and metadata, the metadata including an initial maximum heart rate of the patient study;
displaying, on a user interface, at least some of the strips of ECG data, including a strip of ECG data associated with the initial maximum heart rate; and
overriding the initial maximum heart rate in response to selecting a strip of ECG data associated a lower maximum heart rate.

14. The method of claim 13, wherein the overriding the initial maximum heart rate is performed in response to:
receiving, via the user interface, a user selection of the strip of ECG data comprising the lower maximum heart rate.

15. The method of claim 13, wherein the overriding the initial maximum heart rate comprises overriding all calculated maximum heart rates that are greater than the lower maximum heart rate.

16. The method of claim 13, wherein the metadata includes an initial minimum heart rate, the method further comprising:
overriding the initial minimum heart rate in response to selecting a strip of ECG data comprising a higher minimum heart rate.

17. The method of claim 13, wherein the initial maximum heart rate and the lower maximum heart rate are heart rates calculated during respective most acute sections of the ECG data.

18. The method of claim 17, wherein the respective most acute sections comprise no more than 10 seconds of the ECG data.

19. The method of claim 17, wherein the respective most acute sections comprise no more than 6 beats within the ECG data.

20. The method of claim 17, wherein the heart rates are average heart rates of the most acute sections.
